(19) United States
(12) Patent Application Publication
(10) Pub. No.: US 2025/0290920 A1
(43) Pub. Date: Sep. 18, 2025

(54) TAG/ANTI-TAG SYSTEM FOR SIGNAL AMPLIFICATION IN IMMUNOASSAYS

(71) Applicants: Bender Medsystems GmbH, Vienna (AT); Pierce Biotechnology, Inc., Rockford, IL (US)

(72) Inventors: Markus Miholits, Grosswarasdorf (AT); Susanne Oehler, Wien (AT); Sigrun Badrnya, Wien (AT); Jeremy Weaver, Rockford, IL (US); Brian Webb, Roscoe, IL (US)

(73) Assignees: Bender Medsystems GmbH, Vienna (AT); Pierce Biotechnology, Inc., Rockford, IL (US)

(21) Appl. No.: 19/077,886

(22) Filed: Mar. 12, 2025

Related U.S. Application Data
(60) Provisional application No. 63/564,765, filed on Mar. 13, 2024.

(51) Int. Cl. G01N 33/542; G01N 33/543; G01N 33/573; G01N 33/68

(52) U.S. Cl. CPC G01N 33/542; G01N 33/54386; G01N 33/573; G01N 33/6863; G01N 2333/912; G01N 2470/04

(57) ABSTRACT

Methods and kits for detecting analytes (such as a plurality of analytes) in a sample utilizing signal amplification (such as DMP-based chemical tag signal amplification) are provided.

### CROSS REFERENCE TO RELATED APPLICATIONS

This claims the benefit of U.S. Provisional Application No. 63/564,765, filed Mar. 13, 2024, which is incorporated herein by reference in its entirety.

### FIELD

This disclosure relates to methods and kits for signal amplification in immunoassays, particularly methods and kits that utilize a dimethyl piperidine (DMP)-based tag and labeled antibodies or fragments thereof that are capable of specifically binding to the DMP-based tag.

### SEQUENCE LISTING INCORPORATION BY REFERENCE

The Sequence Listing is submitted as an XML file in the form of the file named “9748-111532-03_Sequence_Listing” (4,502 bytes), which was created on Mar. 12, 2025, which is incorporated by reference herein.

### BACKGROUND

Many current bead-based multiplex immunoassays assays utilize biotin-streptavidin (BT-SA) to amplify the first reporter signal using SA-Phycoerythrin (PE) detection in the green laser reporter channel. For this, a biotinylated detection antibody which binds to its specific target is followed by an incubation with PE-conjugated streptavidin (SA-PE) for signal amplification. Because biotin is relatively small (244.3 Daltons) several molecules can be conjugated randomly (for example, using lysine-based conjugation) to an antibody and so several SA-PE binding partners can attach and amplify the signal. To enable amplified detection of a second independent analyte per bead, an additional affinity binding pair that is discrete from biotin-streptavidin is needed.

### SUMMARY

Provided herein are methods of detecting a plurality of analytes in a sample, the methods including contacting the sample with a solid support including a capture reagent capable of specifically binding the plurality of analytes; contacting the solid support with a plurality of detection antibodies or fragments thereof capable of specifically binding the plurality of analytes, wherein the plurality of detection antibodies or fragment thereof include at least i) a first detection antibody or fragment thereof capable of specifically binding a first analyte and including a first detectable label; and ii) a second detection antibody or fragment thereof capable of specifically binding a second analyte and including a second detectable label; contacting the solid support with a plurality of detection reagents, wherein the plurality of detection reagents include at least: i) a first detection reagent capable of specifically binding to the first detectable label and including a first fluorescent label; and ii) a second detection reagent capable of specifically binding to the second detectable label and including a second fluorescent label, wherein at least one of the first and second detection reagents is an antibody or fragment thereof capable of specifically binding to the first or second detectable label; and detecting the first fluorescent label and the second fluorescent label, thereby detecting the plurality of analytes.

Also provided are methods of detecting an analyte in two or more samples, the method including labeling a plurality of analytes in a first sample with a first detectable label to produce a labeled first sample and labeling a plurality of analytes in a second sample with a second detectable label to produce a labeled second sample; contacting the labeled first sample and the labeled second sample with a solid support including a capture reagent capable of specifically binding at least one of the labeled plurality of analytes; contacting the solid support with a plurality of detection reagents, wherein the plurality of detection reagents include at least i) a first detection reagent capable of specifically binding the first detectable label and including a first fluorescent label; and ii) a second detection reagent capable of specifically binding the second detectable label and including a second fluorescent label, wherein at least one of the first and second detection reagents is an antibody or fragment thereof capable of specifically binding to the first or second detectable label; and detecting the first fluorescent label and the second fluorescent label, thereby detecting the analyte from the first sample and the second sample.

Also provided herein are kits including a solid support including a capture reagent capable of specifically binding an analyte (such as a protein of interest), a first detection antibody or fragment thereof capable of specifically binding the analyte and including a first detectable label, a second detection antibody or fragment thereof capable of specifically binding the analyte (such as an alternative form of the analyte, such as a phosphorylated form of the analyte) and including a second detectable label, a first detection reagent capable of specifically binding to the first detectable label and including a first fluorescent label, and a second detection reagent capable of specifically binding to the second detectable label and including a second fluorescent label, wherein at least one of the first detection reagent and the second detection reagent is an antibody or a fragment thereof capable of specifically binding to the first or second detectable label.

The foregoing and other features of this disclosure will become more apparent from the following detailed description of several aspects which proceeds with reference to the accompanying figures.

### BRIEF DESCRIPTION OF THE DRAWINGS

FIG. 1 is a schematic diagram illustrating exemplary aspects of the disclosed methods. A solid support (such as a bead) is coated with a capture reagent (in this case, an antibody) that specifically binds to an analyte of interest (such as a protein). A first detection antibody binds to one form of the analyte (such as total protein, regardless of phosphorylation state) and a second detection antibody binds to another form of the analyte (such as a phosphorylated form of the protein). The first detection antibody includes a first detectable label (such as a DMP-based chemical tag), and the first detectable label is bound by a first detection reagent including a first fluorescent label. The second detection antibody includes a second detectable label (such as biotin), and the second detectable label is bound by a second detection reagent including a second fluorescent label. In this example, the first detection reagent is an antibody that binds to the DMP-based chemical tag and the first fluorescent label is BV421, and the second detection reagent is streptavidin and the second fluorescent label is phycoerythrin.

FIG. 2 is a schematic diagram showing additional exemplary aspects of the disclosed methods. In this example, a reference sample is labeled with a first detectable label (such as biotin) and a test sample is labeled with a second detectable label (such as a DMP-based chemical tag). The test sample and reference sample are mixed, and contacted with a solid support (such as a bead) that is coated with a capture reagent (in this case, an antibody) that specifically binds to an analyte of interest (such as a protein). The first detectable label is bound by a first detection reagent including a first fluorescent label. The second detectable label is bound by a second detection reagent including a second fluorescent label. In this example, the first detection reagent is streptavidin and the first fluorescent label is phycoerythrin, and the second detection reagent is an antibody that binds to the DMP-based chemical tag and the second fluorescent label is BV421.

FIGS. 3A-3C are graphs showing detection of human IL-31 (FIG. 3A), IL2 (FIG. 3B), or IFNγ (FIG. 3C) with the reference biotin-streptavidin amplification system using biotinylated detection antibody followed by streptavidin-phycoerythrin amplification (BT), DMP-based chemical tag system using a detection antibody conjugated to the DMP-based chemical tag followed by amplification with a phycoerythrin-conjugated anti-tag antibody (TMT), or detection with a phycoerythrin-conjugated detection antibody without amplification (PE). The amount of detection antibody relative to BT (1×) is shown.

FIG. 4 is a graph showing detection of human IFNγ with BT or TMT detection as described in FIGS. 2A and 2B, respectively, or with a digoxigenin-conjugated IFNγ detection antibody followed by phycoerythrin-conjugated anti-digoxigenin secondary antibody (DIG). The amount of detection antibody relative to BT (1×) is shown.

FIG. 5 shows standard curves for the indicated signaling proteins (total and phosphorylated forms).

FIGS. 6A-6B illustrate phospho-specificity by loss of signal from Akt[pS473] (FIG. 6B), but not Akt total (FIG. 6A) following treatment with lambda phosphatase.

FIG. 7 shows phospho-target levels from U87MG cells normalized to total protein and shown relative to untreated cells. Treatment with wortmannin reduced phospho-target levels, and these recovered following treatment with EGFR. FIGS. 8A-8H show correlation of “classic” 8plex for either total or phosphorylated signaling proteins as compared to the dual reporter (DR) 2×8plex panel described in Example 3.

FIGS. 9A-9B show performance comparison of protein analysis using western blot (FIG. 9B) and Human Akt Pathway Dual Reporter Panel, 2×8-plex (FIG. 9A) showing PRAS40[pT246] levels (40 kDa) in lysed NIH-3T3 cells. Equal amounts of protein (10 μg) were used for each assay.

FIGS. 10A-10B show comparison of analytical sensitivity of Human Akt Pathway Dual Reporter Panel, 2×8plex (FIG. 10B) and western blot (FIG. 10A) using a recombinant protein for CREB[pS133].

FIGS. 11A-11B show comparison of analytical sensitivity of Human Akt Pathway Dual Reporter Panel, 2×8plex (FIG. 11B) and western blot (FIG. 11A) for CREB[pS133] using Jurkat cell lysate.

### SEQUENCE LISTING

The nucleic and amino acid sequences listed herein and in the accompanying sequence listing are shown using standard letter abbreviations for nucleotide bases and amino acids. Only one strand of each nucleic acid sequence is shown, but the complementary strand is understood as included by any reference to the displayed strand.
SEQ ID NO: 1 is the amino acid sequence of a histidine (His) tag: HHHHHH
SEQ ID NO: 2 is the amino acid sequence of a FLAG tag: DYKDDDDK
SEQ ID NO: 3 is the amino acid sequence of a Myc tag: EQKLISEEDL
SEQ ID NO: 4 is the amino acid sequence of a hemagglutinin tag: EPEDVPDYA

### DETAILED DESCRIPTION

Streptavidin-biotin interaction is one of the strongest non-covalent interactions in nature. This affinity pair has been exploited in many applications including ELISA, protein and nucleic acid detection and purification, and immobilization of biomolecules on surfaces. It is also the standard amplification method in bead-based multiplex immunoassays (such as Invitrogen ProcartaPlex assays). Introduction of an additional laser and a second reporter channel (for example, Luminex™ xMAP™ INTELLIFLEX system) enables data acquisition for two parameters per bead simultaneously. As a result, an alternative amplification system was needed which can in some examples be used simultaneously with biotin-streptavidin and which enables similar signal amplification. Many attempts have been made to create different high affinity receptor-ligand complexes in place of biotin-streptavidin. While synthetic receptor-ligand complexes often display lower affinity and selectivity in aqueous solution, other natural complexes with comparable affinity to biotin-streptavidin affinity complex additionally need to fulfill several other characteristics: small size to enable higher degree of labeling, easily attached to antibodies, stable, very soluble, and resistant to proteases. Potential background issues resulting from naturally occurring sources of the used components for signal amplification also should be avoided.

In some aspects, disclosed herein are methods utilizing a DMP-based chemical tag and corresponding antibody that fulfills these characteristics. The DMP-based chemical tag is an artificial epitope that exhibits lower non-specific binding and background than is provided by other systems. In addition, the anti-DMP-based tag antibody is highly specific and has high affinity. Finally, this system can be used together with biotin-streptavidin without interference.

### I. Methods of Detecting Analytes Utilizing Signal Amplification

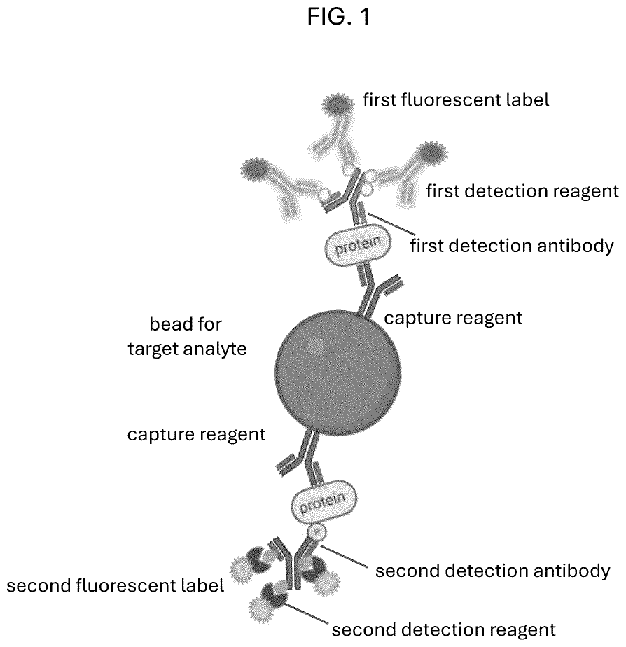

Provided herein are methods of detecting analytes (such as a plurality of analytes) in a sample utilizing signal amplification (such as DMP-based signal amplification). In some aspects, the methods include contacting a sample with a solid support including a capture reagent capable of specifically binding one or more analytes; contacting the solid support with a plurality of detection antibodies or fragments thereof capable of specifically binding the one or more analytes, wherein the plurality of detection antibodies or fragment thereof include at least a first detection antibody or fragment thereof capable of specifically binding a first analyte and including a first detectable label, and a second detection antibody or fragment thereof capable of specifically binding a second analyte and including a second detectable label; contacting the solid support with a plurality of detection reagents, wherein the plurality of detection reagents include at least a first detection reagent capable of specifically binding to the first detectable label and including a first fluorescent label, and a second detection reagent capable of specifically binding to the second detectable label and including a second fluorescent label, wherein at least one of the first and second detection reagents is an antibody or fragment thereof capable of specifically binding to the first or second detectable label; and detecting the first fluorescent label and the second fluorescent label, thereby detecting the one or more analytes. An exemplary aspect is illustrated in FIG. 1.

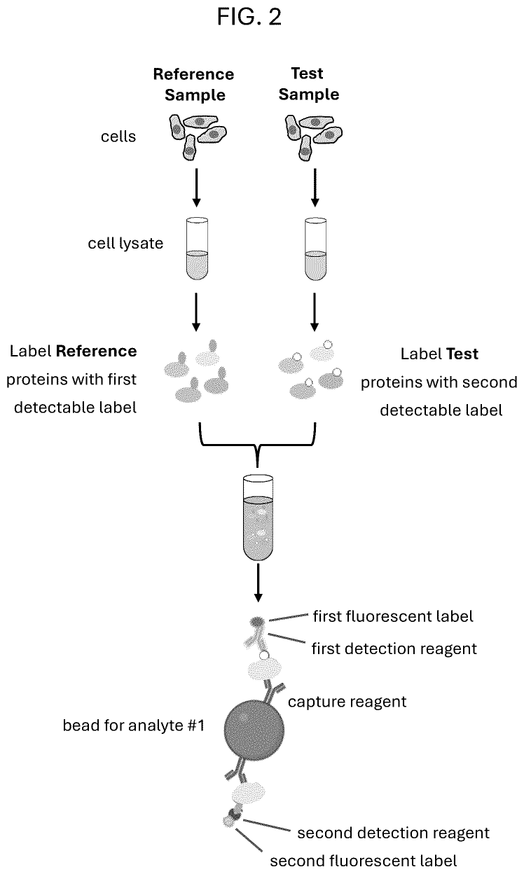

In other aspects, provided herein is a method of detecting an analyte in two or more samples, the method including labeling a plurality of analytes in a first sample with a first detectable label to produce a labeled first sample and labeling a plurality of analytes in a second sample with a second detectable label to produce a labeled second sample; contacting the labeled first sample and the labeled second sample with a solid support including a capture reagent capable of specifically binding at least one of the plurality of labeled analytes; contacting the solid support with a plurality of detection reagents, wherein the plurality of detection reagents comprise at least a first detection reagent capable of specifically binding the first detectable label and including a first fluorescent label and a second detection reagent capable of specifically binding the second detectable label and including a second fluorescent label, wherein at least one of the first and second detection reagents is an antibody or fragment thereof capable of specifically binding to the first or second detectable label; and detecting the first fluorescent label and the second fluorescent label, thereby detecting the analyte from the first sample and the second sample. An exemplary aspect is illustrated in FIG. 2. In some examples, the method further includes comparing a level of the first fluorescent label and the second fluorescent label, thereby detecting a differential amount of the first analyte and the second analyte between the first and second samples. In additional examples, the first sample is a reference sample and the second sample is a test sample, for example, the first sample may be a control or untreated sample and the second sample may be a test or treated sample. In other examples, the first sample may be a sample from a normal or healthy subject or tissue and the second sample may be a sample from a subject with a disease or disorder (such as a subject with a tumor).

In some aspects of the disclosed methods, the capture reagent is an antibody or a fragment thereof that is capable of specifically binding one or more analytes of interest. Non-limiting examples of antibodies include, for example, intact immunoglobulins and variants and fragments thereof that retain binding affinity for an analyte of interest. Examples of antigen binding fragments include but are not limited to Fv, Fab, Fab′, Fab′-SH, F(ab′)₂, diabodies, linear antibodies, single-chain antibody molecules (e.g. scFv), and multi-specific antibodies formed from antibody fragments. Antibody fragments include antigen binding fragments either produced by the modification of whole antibodies or those synthesized de novo using recombinant DNA methodologies. Antibodies also include genetically engineered forms, such as chimeric antibodies (such as humanized murine antibodies) and heteroconjugate antibodies (such as bispecific antibodies). In some examples, the capture reagent is an intact monoclonal or polyclonal immunoglobulin.

In other examples, the capture reagent is a non-antibody capture reagent capable of specifically binding one or more analytes of interest. In some examples, the non-antibody capture reagent is an aptamer. Aptamers are an oligonucleotide or peptide molecule that binds to a specific analyte of interest. In some examples, aptamers are short, single-stranded DNA or RNA (ssDNA or ssRNA) molecules that can selectively bind to a specific analyte, including proteins, peptides, carbohydrates, small molecules, toxins, cells, and/or viruses. Aptamers can assume a variety of shapes due to their tendency to form secondary structures, such as helices and single-stranded loops (for example, a hairpin).

In some aspects of the disclosed methods, the one or more analytes are proteins or a portion thereof. In other aspects, the one or more analytes are nucleic acids, lipids, or carbohydrates (e.g. glycans). In some examples, the disclosed methods can be used to detect a plurality of analytes, such as two or more analytes (such as about 2-500 analytes, for example, about 2-16, about 8-64, about 20-80, about 50-100, about 75-200, or about 200-500). In some aspects, the plurality of analytes include two or more forms of the same protein, such as differentially modified forms of a protein. In some examples, the capture reagent is capable of specifically binding to the “total” protein (for example, regardless of modification state), the first detection antibody is also capable of specifically binding to “total” protein, and the second detection antibody is capable of specifically binding to a particular modified form of the same protein (such as a post-translationally modified form of the protein). Exemplary modifications include, but are not limited to phosphorylation, ubiquitination, glycosylation, S-nitrosylation, methylation, N-acetylation, lipidation, citrullination, and proteolysis. In some examples, an analyte is a protein involved in signal transduction, including, but not limited to a kinase, phosphatase, receptor, adaptor protein, or transcription factor.

In one non-limiting example, a first analyte is “total” protein and a second analyte is a phosphorylated form of the same protein. In this example, the capture reagent is capable of specifically binding to the “total” protein, regardless of phosphorylation state, the first detection antibody is also capable of specifically binding to “total” protein, and the second detection antibody is capable of specifically binding to a particular phosphorylated form of the same protein. Specific examples of total and phosphorylated proteins are provided in Table 1.

In some examples, the plurality of analytes includes total Akt and phospho-Akt (such as Akt[pS473]), total CREB and phospho-CREB (such as CREB[pS133]), total GSK-3β and phospho-GSK-3β (such as GSK-3β[pS9]), total IGF-1R and phospho-IGF-1R (such as IGF-1R[pYpY1135/1136), total mTOR and phospho-mTOR (such as mTOR[pS2448]), total PRAS40 and phospho-PRAS40 (such as PRAS40[pT246]), total p70S6K and phospho-p70S6K (such as p70S6K[pTpS421/424]), total IRS1 and phospho-IRS1 (such as IRS1[pS312]), or any combination of two or more thereof.

The methods described herein utilize a first and second detection antibody; however, alternative binding agents, such as an aptamer that is capable of binding to the first or second detectable label could also be used in the methods. In some aspects of the disclosed methods the first detection antibody or fragment thereof includes a first detectable label and the second detection antibody or fragment thereof includes a second detectable label. The detectable label may be conjugated to the detection antibody or fragment thereof. In some examples, the detection antibody is conjugated to one or more detectable labels (such as 1-20, 5-15, 2-10, or 2-5 detectable labels). The detectable label is a moiety (such as a small molecule, peptide, or protein) that can be specifically bound by a detection reagent. In some examples, the detectable label (such as the first detectable label or the second detectable label) is biotin, a DMP-based chemical tag, digoxigenin, cucurbit[7]uril, or O⁶-benzylguanine (BG). In other examples, the detectable label (such as the first detectable label or the second detectable label) is a His tag (e.g., HHHHHH (SEQ ID NO: 1)), a FLAG tag (e.g., DYKDDDDK (SEQ ID NO: 2), a Myc tag (e.g., EQKLISEEDL (SEQ ID NO: 3), or a hemagglutinin tag (e.g., YPYDVPDYA (SEQ ID NO: 4). In still further examples, the detectable label (such as the first detectable label or the second detectable label) is a fluorescent protein (e.g., green fluorescent protein), HaloTag, or glutathione-S-transferase.

The DMP-based chemical tags of use in the disclosed methods include those described in U.S. Pat. No. 9,835,629, which is incorporated by reference herein in its entirety. In some examples, the DMP-based chemical tag has the structure:

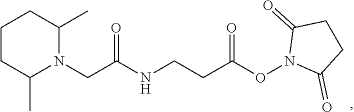 [SMILES: CC1CCCC(C)N1CC(=O)NCCC(=O)ON1C(=O)CCC1=O]

or a derivative thereof.

In some examples, the first detectable label and the second detectable label are different. In some specific examples, the first detectable label is biotin and the second detectable label is a DMP-based chemical tag. In other examples, the first detectable label is a DMP-based chemical tag and the second detectable label is biotin. In further examples, the first detectable label is biotin and the second detectable label is digoxigenin. In still further examples, the first detectable label is digoxigenin and the second detectable label is biotin. In additional examples, the first detectable label is a DMP-based chemical tag and the second detectable label is digoxigenin. In still further examples, the first detectable label is digoxigenin and the second detectable label is a DMP-based chemical tag. One of skill in the art could select other combinations of first and second detectable labels.

In some aspects, the disclosed methods utilize a first detection reagent and a second detection reagent. The first and second detection reagents are capable of specifically binding to the first and second detectable labels, respectively. Each of the first and second detection reagents also include a fluorescent label. The first detection reagent or the second detection reagent may be a partner molecule that is capable of specifically binding the first or second detectable label or may be an antibody or fragment thereof capable of specifically binding the first or second detectable label. At least one of the first and second detection reagents is an antibody or fragment thereof capable of specifically binding to the first or second detectable label.

In some examples, the first detection reagent or the second detection reagent is streptavidin, an antibody or fragment thereof that specific binds a dimethyl piperidine (DMP)-based chemical tag, an antibody or fragment thereof that specifically binds to digoxigenin, an antibody or fragment thereof that specifically binds to cucurbit[7]uril, SNAP tag (O⁶-alkylguanine-DNA alkyltransferase (AGT)), an antibody or fragment thereof that specifically binds to a His tag comprising the amino acid sequence HHHHHH (SEQ ID NO: 1), an antibody or fragment thereof that specifically binds to a FLAG tag comprising the amino acid sequence DYKDDDDK (SEQ ID NO: 2), an antibody or fragment thereof that specifically binds to a Myc tag comprising the amino acid sequence EQKLISEEDL (SEQ ID NO: 3), an antibody or fragment thereof that specifically binds to a hemagglutinin tag comprising the amino acid sequence YPYDVPDYA (SEQ ID NO: 4), an antibody or fragment thereof that specifically binds to a fluorescent protein (such as a green fluorescent protein), an antibody or fragment thereof that specifically binds to HaloTag, or an antibody or fragment thereof that specifically binds to glutathione S-transferase. In some examples, an antibody or fragment thereof that specifically binds to the DMP-based chemical tag is an antibody described in U.S. Pat. No. 9,835,629 (incorporated herein by reference). In some examples, the antibody that specifically binds to the DMP-based chemical tag is TMT monoclonal antibody 25D5 (Thermo Fisher Scientific, Cat. No. 90075). One of ordinary skill in the art can select appropriate first and second detection reagents based on the first and second detectable labels being used.

In some examples, the first detection reagent is streptavidin and the second detection reagent is an antibody or fragment thereof that specifically binds the DMP-based chemical tag (for example, if the first detectable label is biotin and the second detectable label is a DMP-based chemical tag). In other examples, the first detection reagent is an antibody or fragment thereof that specifically binds the DMP-based chemical tag and the second detection reagent is streptavidin (for example, if the first detectable label is the DMP-based chemical tag and the second detectable label is biotin). In further examples, the first detection reagent is streptavidin and the second detection reagent is an antibody or fragment thereof that specifically binds digoxigenin (for example if the first detectable label is biotin and the second detectable label is digoxigenin). In still further examples, the first detection reagent is an antibody or fragment thereof that specifically binds digoxigenin and the second detection reagent is streptavidin (for example, if the first detectable label is digoxigenin and the second detectable label is biotin). In further examples, the first detection reagent is an antibody or fragment thereof that specifically binds a DMP-based chemical tag and the second detection reagent is an antibody or fragment thereof that specifically binds digoxigenin (for example if the first detectable label is a DMP-based chemical tag and the second detectable label is digoxigenin). In still further examples, the first detection reagent is an antibody or fragment thereof that specifically binds digoxigenin and the second detection reagent is an antibody or fragment thereof that specifically binds a DMP-based chemical tag (for example, if the first detectable label is digoxigenin and the second detectable label is a DMP-based chemical tag).

Each of the first and second detection reagents include a fluorescent label, such as a fluorescent label or fluorochrome. The fluorescent label may be conjugated to the detection reagent. In some examples, the first and second fluorescent labels are different, for example, have different excitation and emission spectra. One of skill in the art can select suitable fluorescent labels for use in the disclosed methods for detecting a plurality of analytes.

In some examples, the fluorescent label is phycoerythrin, carboxyfluorescein (FAM), tetrachlorofluorescein (TET), tetramethylrhodamine (TMR), hexachlorofluorescein (HEX), JOE, ROX, CAL Fluor™, Pulsar™, Quasar™, Texas Red™, Cy™ 3 dye, Cy™ 5 dye, 4-acetamido-4′-isothiocyanatostilbene-2,2′disulfonic acid, acridine and derivatives such as acridine and acridine isothiocyanate, 5-(2′-aminoethyl)amino-naphthalene-1-sulfonic acid (EDANS), 4-amino-N-[3-vinylsulfonyl)phenyl]-naphthalimide-3,5 disulfonate (Lucifer Yellow VS), N-(4-anilino-1-naphthyl)-maleimide, anthranilamide, Brilliant Yellow, coumarin and derivatives such as coumarin, 7-amino-4-methylcoumarin (AMC, Coumarin 120), 7-amino-4-trifluoromethylcouluarin (Coumaran 151); cyanosine; 4′,6-diaminidino-2-phenylindole (DAPI); 5′, 5″-dibromopyrogallol-sulfonephthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethyl-amino]naphthalene-1-sulfonyl chloride (DNS, dansyl chloride); 4-(4′-dimethyl-aminophenylazo)benzoic acid (DABCYL); 4-dimethylaminophenylazophenyl-4′-isothiocyanate (DABITC); eosin and derivatives such as eosin and eosin isothiocyanate; erythrosin and derivatives such as erythrosin B and erythrosin isothiocyanate; ethidium; fluorescein and derivatives such as 5-carboxyfluorescein (FAM), 5-(4,6-dichlorotriazin-2-yl)aminofluorescein (DTAF), 2′7′-dimethoxy-4′5′-dichloro-6-carboxyfluorescein (JOE), fluorescein, fluorescein isothiocyanate (FITC), and QFITC (XRITC); fluorescamine; IR144; IR1446; Malachite Green isothiocyanate; 4-methylumbelliferone; ortho cresolphthalein; nitrotyrosine; pararosaniline; Phenol Red; B-phycoerythrin; o-phthaldialdehyde; pyrene and derivatives such as pyrene, pyrene butyrate and succinimidyl 1-pyrene butyrate; Reactive Red 4 (Cibacron® Brilliant Red 3B-A); rhodamine and derivatives such as 6-carboxy-X-rhodamine (ROX), 6-carboxyrhodamine (R6G), lissamine rhodamine B sulfonyl chloride, rhodamine (Rhod), rhodamine B, rhodamine 123, rhodamine X isothiocyanate, sulforhodamine B, sulforhodamine 101 and sulfonyl chloride derivative of sulforhodamine 101 (Texas Red); N,N,N′,N′-tetramethyl-6-carboxyrhodamine (TAMRA); tetramethyl rhodamine; tetramethyl rhodamine isothiocyanate (TRITC); riboflavin; rosolic acid and terbium chelate derivatives, Oregon Green 488, Pacific Blue, Pacific Green, Pacific Orange, Texas Red, Texas Red-X, a NovaFluor® dye, an AlexaFluor® dye, an eFluor® dye, or a quantum dot.

In other examples, the fluorescent label is a polymer dye, such as a Brilliant Violet™ dye (Sirigen, see also U.S. Pat. No. 10,962,546), a Super Bright® dye or Brilliant Ultra Violet™ dye (Thermo Fisher Scientific).

In some examples, one of the first or second fluorescent label is a fluorescent dye that is excited by a blue laser (e.g., about 488-509 nm) or yellow-green laser (e.g., about 550-562 nm). An exemplary fluorescent dye excited by blue or yellow-green laser includes phycoerythrin. In further examples, one of the first or second fluorescent labels is a fluorescent dye that is excited by a violet laser (e.g., about 405 nm). Exemplary fluorescent dyes excited by violet laser include Brilliant Violet™ dyes (e.g., Brilliant Violet™ 421, Brilliant Violet™ 480, Brilliant Violet™ 605, Brilliant Violet™ 650, Brilliant Violet™ 711, or Brilliant Violet™ 786, Sirigen, La Jolla, CA), Super Bright dyes (e.g., Super Bright 436, Super Bright 600, Super Bright 645, Super Bright 702, or Super Bright 786, Invitrogen), eFluor® dyes (e.g., eFluor® 450 or eFluor® 506, Affymetrix). In some non-limiting examples, the first fluorescent label in phycoerythrin and the second fluorescent label is Brilliant Violet™ 421. In other non-limiting examples, the first fluorescent label is Brilliant Violet™ 421 and the second fluorescent label is phycoerythrin. One of ordinary skill in the art can select appropriate first and second fluorescent labels for use in the methods disclosed herein.

In some aspects of the disclosed methods the solid support is a bead, such as a magnetic bead. In other aspects, the solid support is a plate, a slide (such as a glass slide), or a membrane (such as a nitrocellulose membrane). Any substrate to which a capture reagent can be associated (for example, conjugated to) can be utilized.

In some aspects of the disclosed methods, the methods include contacting the sample with a plurality of solid supports, each of which includes a different capture reagent. In some examples, the plurality of solid supports include 2-500 solid supports (for example, 2-16, 8-64, 20-80, 50-100, 75-200, or 200-500 solid supports), each of which includes a different capture reagent. In some examples, the solid support is a bead or the plurality of solid supports is a plurality of beads. In some examples, the bead or plurality of beads are magnetic beads. In additional examples, the plurality of beads may further each include a unique fluorescent dye (e.g., LUMINEX™ beads). In other aspects of the disclosed methods, the plurality of solid supports may be a single solid support including a plurality of regions (such as a plurality of addressable regions), each region including a different capture reagent (such as an array).

In some aspects, the methods include detecting the first fluorescent label and the second fluorescent label. In some examples, the detecting includes utilizing a flow-based detection instrument, wherein the first fluorescent label and the second fluorescent label are detected using excitation by suitable laser(s) and detection of light emitted from the first and second fluorescent labels. In some examples, the flow-based detection instrument is a Luminex XMAP INTELLIFLEX dual reporter instrument. In other examples, the detecting utilizes a flow cytometer, a fluorescence microscope, or a fluorescent plate reader. In some examples, the detection is qualitative, semi-quantitative, or quantitative. In semi-quantitative or quantitative aspects of the methods, a standard curve may be used to determine amounts of the plurality of analytes. In some examples, a standard curve is generated in parallel with the disclosed methods. In other examples, a reference standard curve is used (for example, a previously generated standard curve).

The disclosed methods can be used with any biological sample. In some examples, the biological sample is an in vitro biological sample, such as from a cell culture system. In other examples, the biological sample is from a human or animal subject. In some examples, the human or animal subject is a subject having or suspected of having a disease or disorder. In some examples, the sample includes cells, cell lysate, cell culture supernatant, extracellular vesicles, extracellular vesicle lysate, blood, serum, plasma, cerebrospinal fluid, urine, sputum, bronchoalveolar lavage, saliva, tears, tissue, or tissue homogenate.

Other methods of utilizing the DMP-based chemical tags and antibody or fragment thereof that specifically binds the DMP-based chemical tags are also contemplated. In particular, these tags and antibodies can be utilized in immunoassays where signal amplification is desirable, for example, similarly to uses of biotin and streptavidin. Such methods may include Western blot, ELISA, flow cytometry, and imaging applications. In such applications, a detection antibody or fragment thereof capable of binding to an analyte of interest is labeled with the DMP-based chemical tag. The detection antibody is then detected with an antibody or fragment thereof capable of binding to the DMP-based chemical tag, which antibody is labeled with a fluorescent label or other detectable label (for example, an enzyme such as horseradish peroxidase or alkaline phosphatase). In these examples, the assay may be singleplex (for example, utilizing a solid support with a capture reagent capable of specifically binding one analyte) or multiplex (for example, utilizing multiple solid supports, each solid support including a capture reagent capable of specifically binding to a different analyte). In such examples, appropriate labeled detection antibod(ies) and detection reagents can be selected, depending on whether the assay is singleplex or multiplex.

### II. Kits

Also provided herein are kits, for example, kits that can be utilized with the methods disclosed herein. In some aspects, the kit includes a solid support including a capture reagent capable of specifically binding an analyte (such as a protein of interest), a first detection antibody or fragment thereof capable of specifically binding the analyte and including a first detectable label, a second detection antibody or fragment thereof capable of specifically binding the analyte (such as an alternative form of the analyte, for example, a post-translationally modified form of the analyte, such as a form of the analyte with differential phosphorylation, ubiquitination, glycosylation, S-nitrosylation, methylation, acetylation, lipidation, citrullination, or a proteolytic fragment) and including a second detectable label, a first detection reagent capable of specifically binding to the first detectable label and including a first fluorescent label, and a second detection reagent capable of specifically binding to the second detectable label and including a second fluorescent label, wherein at least one of the first detection reagent and second detection reagent is an antibody or a fragment thereof capable of specifically binding to the first or second detectable label.

In some examples, the capture reagent is an antibody or a fragment thereof that is capable of specifically binding one or more analytes of interest. Non-limiting examples of antibodies include, for example, intact immunoglobulins and variants and fragments thereof that retain binding affinity for an analyte of interest. Examples of antigen binding fragments include but are not limited to Fv, Fab, Fab′, Fab′-SH, F(ab′)₂, diabodies, linear antibodies, single-chain antibody molecules (e.g. scFv), and multi-specific antibodies formed from antibody fragments. In other examples, the capture reagent is a non-antibody capture reagent capable of specifically binding one or more analytes of interest. In some examples, the non-antibody capture reagent is an aptamer.

In some aspects, the first detection antibody or fragment thereof includes a first detectable label and the second detection antibody or fragment thereof includes a second detectable label. The detectable label is a moiety (such as a small molecule, peptide, or protein) that can be specifically bound by a detection reagent. In some examples, the detectable label (such as the first detectable label or the second detectable label) is biotin, a DMP-based chemical tag, digoxigenin, cucurbit[7]uril, or O⁶-benzylguanine. In other examples, the detectable label (such as the first detectable label or the second detectable label) is a His tag (e.g., HHHHHH (SEQ ID NO: 1)), a FLAG tag (e.g., DYKDDDDK (SEQ ID NO: 2), a Myc tag (e.g., EQKLISEEDL (SEQ ID NO: 3), or a hemagglutinin tag (e.g., YPYDVPDYA (SEQ ID NO: 4). In still further examples, the detectable label (such as the first detectable label or the second detectable label) is a fluorescent protein (such as green fluorescent protein), HaloTag, or glutathione-S-transferase.

The DMP-based chemical tags of use in the disclosed methods include those described in U.S. Pat. No. 9,835,629, which is incorporated by reference herein in its entirety. In some examples, the DMP-based chemical tag has the structure:

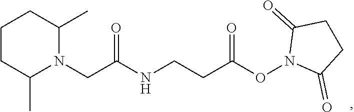 [SMILES: CC1CCCC(C)N1CC(=O)NCCC(=O)ON1C(=O)CCC1=O]

or a derivative thereof.

In some examples, the first detectable label and the second detectable label are different. In some specific examples, the first detectable label is biotin and the second detectable label is a DMP-based chemical tag. In other examples, the first detectable label is a DMP-based chemical tag and the second detectable label is biotin. In further examples, the first detectable label is biotin and the second detectable label is digoxigenin. In still further examples, the first detectable label is digoxigenin and the second detectable label is biotin. One of skill in the art could select other combinations of first and second detectable labels.

In some aspects, the first and second detection reagents are capable of specifically binding to the first and second detectable labels, respectively. Each of the first and second detection reagents also include a fluorescent label. The first detection reagent or the second detection reagent may be a partner molecule that is capable of specifically binding the first or second detectable label or may be an antibody or fragment thereof capable of specifically binding the first or second detectable label. At least one of the first and second detection reagents is an antibody or fragment thereof capable of specifically binding to the first or second detectable label.

In some examples, the first detection reagent or the second detection reagent is streptavidin, an antibody or fragment thereof that specific binds a dimethyl piperidine (DMP)-based chemical tag, an antibody or fragment thereof that specifically binds to digoxigenin, an antibody or fragment thereof that specifically binds to cucurbit[7]uril, SNAP tag (O⁶-alkylguanine-DNA alkyltransferase (AGT)), an antibody or fragment thereof that specifically binds to a His tag comprising the amino acid sequence HHHHHH (SEQ ID NO: 1), an antibody or fragment thereof that specifically binds to a FLAG tag comprising the amino acid sequence DYKDDDDK (SEQ ID NO: 2), an antibody or fragment thereof that specifically binds to a Myc tag comprising the amino acid sequence EQKLISEEDL (SEQ ID NO: 3), an antibody or fragment thereof that specifically binds to a hemagglutinin tag comprising the amino acid sequence YPYDVPDYA (SEQ ID NO: 4), an antibody or fragment thereof that specifically binds to a fluorescent protein (such as a green fluorescent protein), an antibody or fragment thereof that specifically binds HaloTag, or an antibody or fragment thereof that specifically binds to glutathione S-transferase. In some examples, an antibody or fragment thereof that specifically binds to the DMP-based chemical tag is an antibody described in U.S. Pat. No. 9,835,629 (incorporated herein by reference). In some examples, the antibody that specifically binds to the DMP-based chemical tag is TMT monoclonal antibody 25D5 (Thermo Fisher Scientific, Cat. No. 90075). One of ordinary skill in the art can select appropriate first and second detection reagents based on the first and second detectable labels being used.

In some examples, the first detection reagent is streptavidin and the second detection reagent is an antibody or fragment thereof that specifically binds the DMP-based chemical tag (for example, if the first detectable label is biotin and the second detectable label is a DMP-based chemical tag). In other examples, the first detection reagent is an antibody or fragment thereof that specifically binds the DMP-based chemical tag and the second detection reagent is streptavidin (for example, if the first detectable label is the DMP-based chemical tag and the second detectable label is biotin). In further examples, the first detection reagent is streptavidin and the second detection reagent is an antibody or fragment thereof that specifically binds digoxigenin (for example if the first detectable label is biotin and the second detectable label is digoxigenin). In still further examples, the first detection reagent is an antibody or fragment thereof that specifically binds digoxigenin and the second detection reagent is streptavidin (for example, if the first detectable label is digoxigenin and the second detectable label is biotin).

Each of the first and second detection reagents includes a fluorescent label, such as a fluorescent label or fluorochrome. The fluorescent label may be conjugated to the detection reagent. In some examples, the detection reagent and the fluorescent label are linked together, for example, linked together by a covalent bond. In some examples, the first and second fluorescent labels are different, for example, have different excitation and emission spectra. One of skill in the art can select suitable fluorescent labels for use in the disclosed methods for detecting a plurality of analytes.

In some examples, the fluorescent label is phycoerythrin, carboxyfluorescein (FAM), tetrachlorofluorescein (TET), tetramethylrhodamine (TMR), hexachlorofluorescein (HEX), JOE, ROX, CAL Fluor™, Pulsar™, Quasar™, Texas Red™, Cy™ 3 dye, Cy™ 5 dye, 4-acetamido-4′-isothiocyanatostilbene-2,2′disulfonic acid, acridine and derivatives such as acridine and acridine isothiocyanate, 5-(2′-aminoethyl)amino-naphthalene-1-sulfonic acid (EDANS), 4-amino-N-[3-vinylsulfonyl)phenyl]-naphthalimide-3,5 disulfonate (Lucifer Yellow VS), N-(4-anilino-1-naphthyl)-maleimide, anthranilamide, Brilliant Yellow, coumarin and derivatives such as coumarin, 7-amino-4-methylcoumarin (AMC, Coumarin 120), 7-amino-4-trifluoromethylcouluarin (Coumaran 151); cyanosine; 4′,6-diaminidino-2-phenylindole (DAPI); 5′, 5″-dibromopyrogallol-sulfonephthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethyl-amino]naphthalene-1-sulfonyl chloride (DNS, dansyl chloride); 4-(4′-dimethyl-aminophenylazo)benzoic acid (DABCYL); 4-dimethylaminophenylazophenyl-4′-isothiocyanate (DABITC); eosin and derivatives such as eosin and eosin isothiocyanate; erythrosin and derivatives such as erythrosin B and erythrosin isothiocyanate; ethidium; fluorescein and derivatives such as 5-carboxyfluorescein (FAM), 5-(4,6-dichlorotriazin-2-yl)aminofluorescein (DTAF), 2′7′-dimethoxy-4′5′-dichloro-6-carboxyfluorescein (JOE), fluorescein, fluorescein isothiocyanate (FITC), and QFITC (XRITC); fluorescamine; IR144; IR1446; Malachite Green isothiocyanate; 4-methylumbelliferone; ortho cresolphthalein; nitrotyrosine; pararosaniline; Phenol Red; B-phycoerythrin; o-phthaldialdehyde; pyrene and derivatives such as pyrene, pyrene butyrate and succinimidyl 1-pyrene butyrate; Reactive Red 4 (Cibacron® Brilliant Red 3B-A); rhodamine and derivatives such as 6-carboxy-X-rhodamine (ROX), 6-carboxyrhodamine (R6G), lissamine rhodamine B sulfonyl chloride, rhodamine (Rhod), rhodamine B, rhodamine 123, rhodamine X isothiocyanate, sulforhodamine B, sulforhodamine 101 and sulfonyl chloride derivative of sulforhodamine 101 (Texas Red); N,N,N′,N′-tetramethyl-6-carboxyrhodamine (TAMRA); tetramethyl rhodamine; tetramethyl rhodamine isothiocyanate (TRITC); riboflavin; rosolic acid and terbium chelate derivatives, Oregon Green 488, Pacific Blue, Pacific Green, Pacific Orange, Texas Red, Texas Red-X, a NovaFluor® dye, an AlexaFluor® dye, an eFluor® dye, or a quantum dot.

In other examples, the fluorescent label is a polymer dye, such as a Brilliant Violet™ dye (Sirigen, see also U.S. Pat. No. 10,962,546), a Super Bright® dye or Brilliant Ultra Violet™ dye (Thermo Fisher Scientific).

In some examples, one of the first or second fluorescent label is a fluorescent dye that is excited by a blue laser (e.g., about 488-509 nm) or yellow-green laser (e.g., about 550-562 nm). An exemplary fluorescent dye excited by blue or yellow-green laser includes phycoerythrin. In further examples, one of the first or second fluorescent labels is a fluorescent dye that is excited by a violet laser (e.g., about 405 nm). Exemplary fluorescent dyes excited by violet laser include Brilliant Violet™ dyes (e.g., Brilliant Violet™ 421, Brilliant Violet™ 480, Brilliant Violet™ 605, Brilliant Violet™ 650, Brilliant Violet™ 711, or Brilliant Violet™ 786, Sirigen, La Jolla, CA), Super Bright dyes (e.g., Super Bright 436, Super Bright 600, Super Bright 645, Super Bright 702, or Super Bright 786, Invitrogen), eFluor® dyes (e.g., eFluor® 450 or eFluor® 506, Affymetrix). In some non-limiting examples, the first fluorescent label in phycoerythrin and the second fluorescent label is Brilliant Violet™ 421. In other non-limiting examples, the first fluorescent label is Brilliant Violet™ 421 and the second fluorescent label is phycoerythrin. One of ordinary skill in the art can select appropriate first and second fluorescent labels for use in the methods disclosed herein.

In some aspects the kit includes a plurality of solid supports, each of which includes a different capture reagent. In some examples, the plurality of solid supports include 2-500 solid supports (for example, 2-16, 8-64, 20-80, 50-100, 75-200, or 200-500 solid supports), each of which includes a different capture reagent. In some examples, the solid support is a bead or the plurality of solid supports is a plurality of beads. In some examples, the bead or plurality of beads are magnetic beads. In additional examples, the plurality of beads may further each include a unique fluorescent dye (e.g., LUMINEX™ beads).

In some aspects, the disclosed kits may include one or more additional components, such as one or more buffers, enzymes, standards, tubes, and/or plates.

In some examples, the kit includes a plurality of solid supports (such as a plurality of beads), each including a different capture reagent. In one example, the kit includes a plurality of beads each including a different capture reagent, wherein the capture reagents include an antibody or fragment thereof capable of specifically binding to Akt, an antibody or fragment thereof capable of specifically binding to CREB, an antibody or fragment thereof capable of specifically binding to GSK-3β, an antibody or fragment thereof capable of specifically binding to IGF-1R, an antibody or fragment thereof capable of specifically binding to mTOR, an antibody or fragment thereof capable of specifically binding to PRAS40, an antibody or fragment thereof capable of specifically binding to p70S6K, and an antibody or fragment thereof capable of specifically binding to IRS1. In additional examples, the kit further includes a detection antibody capable of specifically binding to total Akt and including a first detectable label, a detection antibody capable of specifically binding to Akt[pS473] and including a second detectable label, a detection antibody capable of specifically binding to total CREB and including a first detectable label, a detection antibody capable of specifically binding to CREB[pS133] and including a second detectable label, a detection antibody capable of specifically binding to total GSK-3β and including a first detectable label, a detection antibody capable of specifically binding to GSK-3β[pS9] and including a second detectable label, a detection antibody capable of specifically binding to total IGF-1R and including a first detectable label, a detection antibody capable of specifically binding to IGF-1R[pYpY1135/1136] and including a second detectable label, a detection antibody capable of specifically binding to total mTOR and including a first detectable label, a detection antibody capable of specifically binding to mTOR[pS2448] and including a second detectable label, a detection antibody capable of specifically binding to total PRAS40 and including a first detectable label, a detection antibody capable of specifically binding to PRAS40[pT246] and including a second detectable label, a detection antibody capable of specifically binding to total p70S6K and including a first detectable label, a detection antibody capable of specifically binding to p70S6K[pTpS421/424] and including a second detectable label, a detection antibody capable of specifically binding to total IRS1 and including a first detectable label, and a detection antibody capable of specifically binding to IRS1[pS312] and including a second detectable label. In some examples, the first detectable label is biotin and the first detection reagent is streptavidin and the second detectable label is a DMP-based chemical tag and the second detection reagent is an antibody or fragment thereof capable of specifically binding to the DMP-based chemical tag. In further examples, the first fluorescent label is phycoerythrin and the second fluorescent label is a polymer dye (e.g., Brilliant Violet™ 421).

### EXAMPLES

The following examples are provided to illustrate particular features of certain aspects of the disclosure, but the scope of the claims should not be limited to those features exemplified.

### Example 1

### Development of DMP-Based Chemical Tag Immunoassay

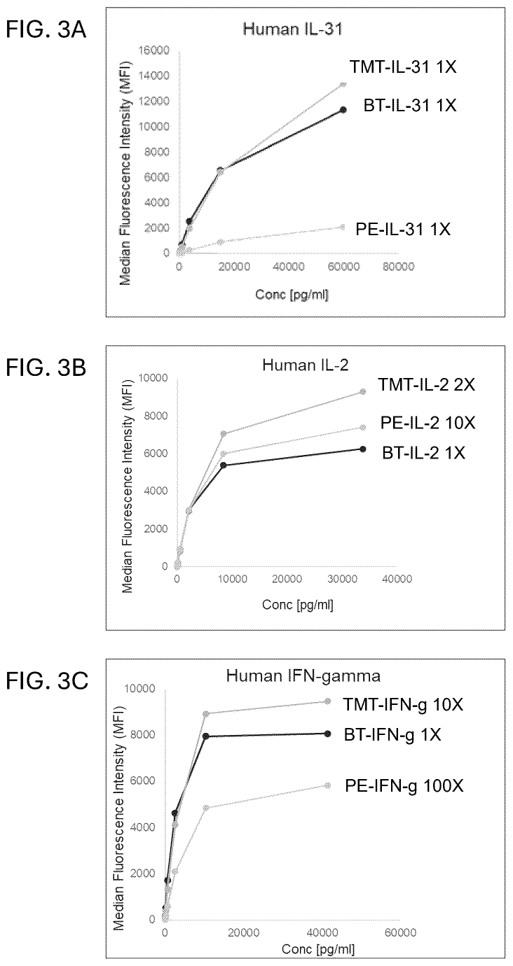

Assays for human IL-31, human IL-2m and human IFNγ were performed according to the following protocol. First, 50 μL of capture beads was added to each well of a 96-well flat bottom plate. After washing of capture beads, 25 μL of Universal Assay Buffer (1×) was added to each well, followed by 25 μL of prepared standards (4-fold serial dilution of respective calibrator protein in Universal Assay Buffer (1×)). For background wells, 50 μL of Universal Assay Buffer (1×) was added. The plate was sealed and incubated for 120 min at room temperature in the dark on a plate shaker at 600 rpm. After incubation, the plate was washed twice (1× wash buffer) and incubated for 30 min at room temperature in the dark on a plate shaker at 600 rpm with 25 μL of either biotin-(BT) conjugated detection antibody, TMT-conjugated detection antibody, or directly Phycoerythrin (PE)-labeled detection antibody at concentrations (1×-100×) as indicated in FIG. 3A-C. Following another wash step to remove unbound antibody in the case of BT-conjugated detection antibody used, 50 μL of Streptavidin-PE (SAPE) solution was added to each well and incubated for 30 min at room temperature in the dark on a plate shaker at 600 rpm. In the case of TMT-conjugated detection antibody used, 50 L of PE-labeled anti-TMT antibody was added to each well and incubated for 30 min at room temperature in the dark on a plate shaker at 600 rpm. After washing the plate twice, 120 μL of Reading Buffer was added and the sealed plate incubated for 5 min at room temperature in the dark on a plate shaker at 600 rpm. Data was acquired on a Luminex™ xMAP™ INTELLIFLEX instrument.

Detection of three cytokines with biotin/streptavidin or DMP-based tag/anti-tag (TMT/anti-TMT) signal amplification was compared with unamplified assay. The TMT/anti-TMT system achieved reference signal with equal to maximally 10-fold higher input of detection antibody (dependent on the assay and its detection antibody), while an assay system without amplification system required (directly labeled) detection antibody of up to 100-fold or even higher (FIGS. 3A-C).

### Example 2

### Development of Digoxigenin/Anti-Digoxigenin Immunoassay

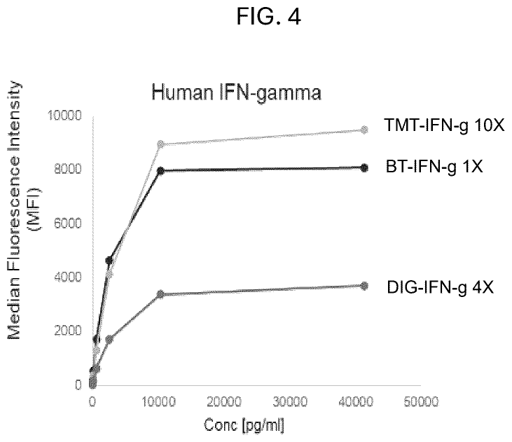

Multiple studies have shown sensitivity of digoxigenin (DIG) labeled protein to be higher than biotin-tagged antibodies in Western Blot, ELISA or immunocytochemistry experiments. Digoxigenin is a secondary metabolite isolated from digitalis plants—no background issues are expected in other biological materials. DIG/anti-DIG-antibody was evaluated as an affinity binding pair for signal amplification on the Luminex-based assay platform using the human IFN-gamma ProcartaPlex assay with biotinylated (BT) detection antibody followed by SA-PE for signal amplification as a refence. With a DIG-conjugated IFNγ detection antibody followed by its PE-conjugated anti-DIG secondary antibody, the refence signal could not be reached even with a 40-fold increased antibody concentration. On the other hand, the TMT-conjugated detector could be easily adjusted (10×) to reach reference signal (FIG. 4).

### Example 3

### Akt Pathway Dual Reporter Panel 2×8-plex

Luminex beads were coupled with target specific pan-capture antibodies which bind the signaling proteins independent of their phosphorylation state. Panels for separate multiplex analysis of total or phospho-signaling proteins were established with either non phospho-specific or phospho-specific detection antibodies both using PE as the dye for reporter channel 1, suitable for all Luminex instruments. For the Dual Reporter Panel, the phospho-specific detectors used a new violet dye for reporter channel 2 on the INTELLIFLEX DR-SE combined with the pan-detector read in the PE reporter channel 1 to simultaneously quantify total and phosphorylated protein in the Human Akt Pathway Dual Reporter Panel 2×8-plex (Table 1).

TABLE 1
Human Akt Pathway Dual Reporter Panel 2 × 8-plex

| Analyte | Bead Region | S1 U/ml |
|---|---|---|
| Akt[pS473] | 47 | 5000 |
| CREB[pS133] | 43 | 500 |
| GSK-3β[pS9] | 56 | 200 |
| IGF-1R[pYpY1135/1136] | 34 | 1000 |
| mTOR[pS2448] | 26 | 200 |
| PRAS40[pT246] | 42 | 4000 |
| p70S6K[pTpS421/424] | 27 | 500 |
| IRS1 [pS312] | 37 | 500 |
| Akt total | 47 | 20000 |
| CREB total | 43 | 6000 |
| GSK-3β total | 56 | 2000 |
| IGF-1R total | 34 | 1000 |
| mTOR total | 26 | 10000 |
| PRAS40 total | 42 | 10000 |
| p70S6K total | 27 | 2000 |
| IRS1 total | 37 | 3000 |

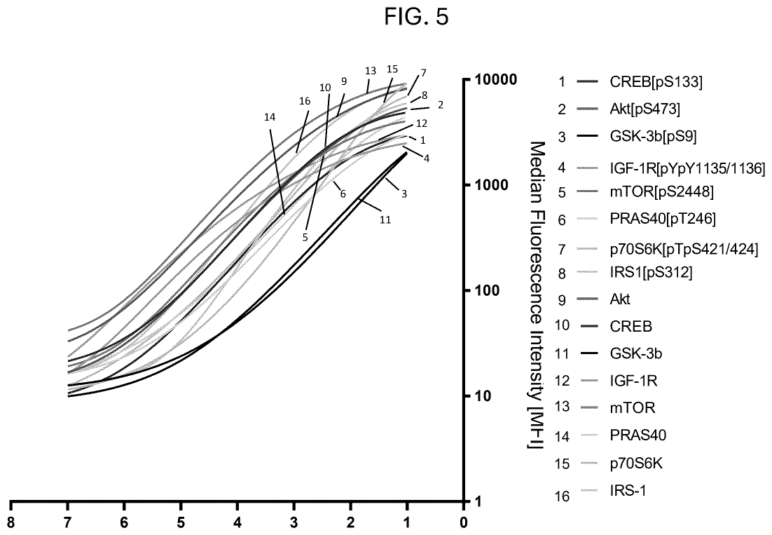

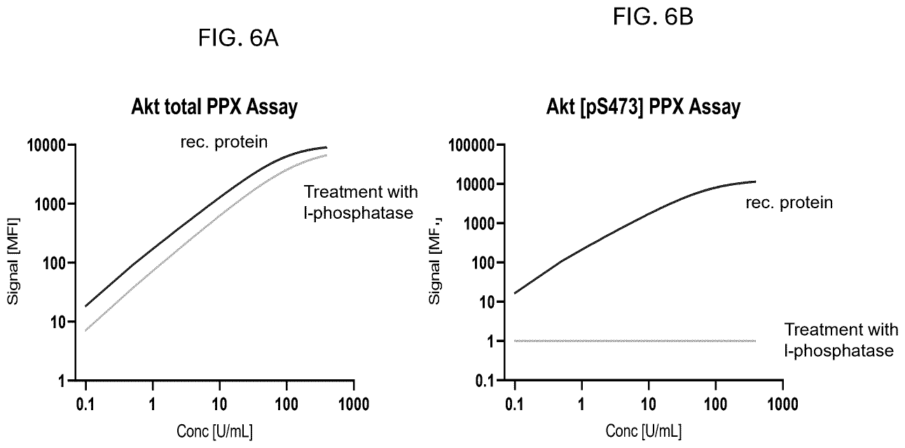

Standard curves with phosphorylated proteins enable relative quantification (U/ml) for all signaling proteins (total and phosphorylated) (FIG. 5). These recombinant proteins were also used to confirm phospho-specificity. Either post-translationally modified active proteins dephosphorylated with lambda phosphatase were used, which resulted in loss or reduction of signal in the p-assay whereas the signal of the dephosphorylated protein remained unchanged in the total assay (data shown for Akt; FIGS. 6A and 6B) or phospho-specificity was verified by analyzing phospho-standard proteins before and after phosphorylation (data not shown).

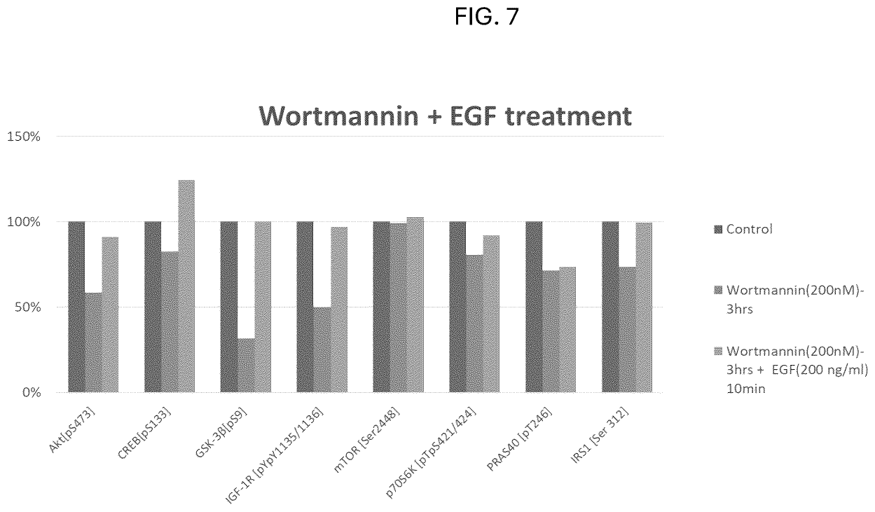

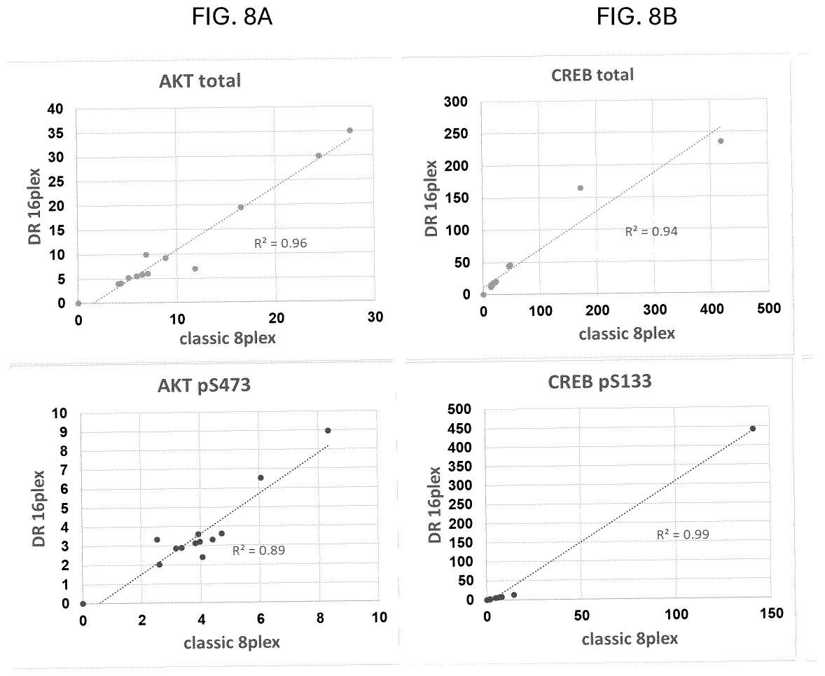

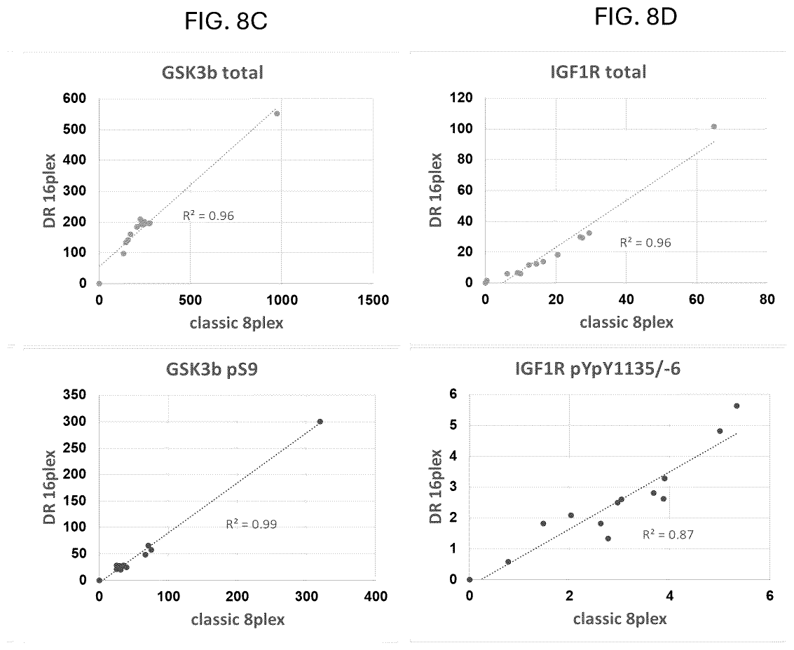

U87MG cells, a cell line with mutation of PTEN, thus expressing high AKT levels, were treated with Wortmannin or Wortmannin+EGF. Phospho-target levels were normalized to total protein levels and are shown relative to untreated levels (FIG. 7). A reduction due to Wortmannin treatment and recovery of levels after EGF treatment could be shown.

The conventional separate 8-plex panels for either total or phosphorylated signaling proteins were run side-by-side to the new Dual Reporter 2×8-plex panel to confirm consistency and scalability (FIGS. 8A-8H).

### Example 4

### Comparison of Sensitivity of Assay With Western Blot

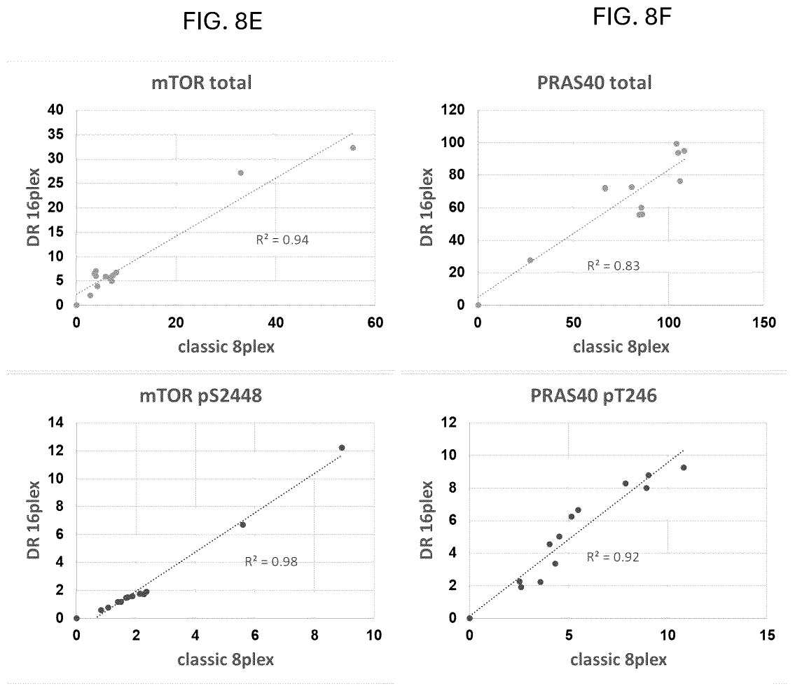

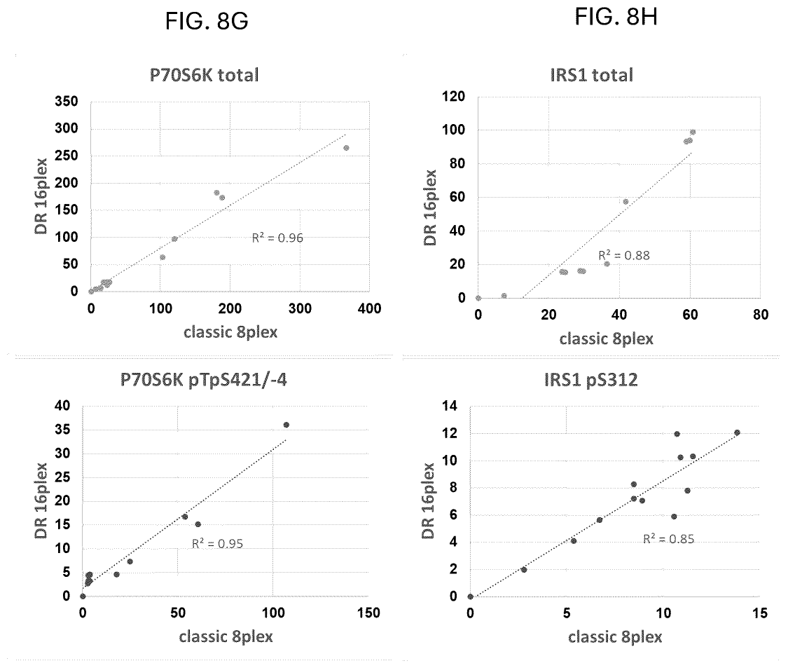

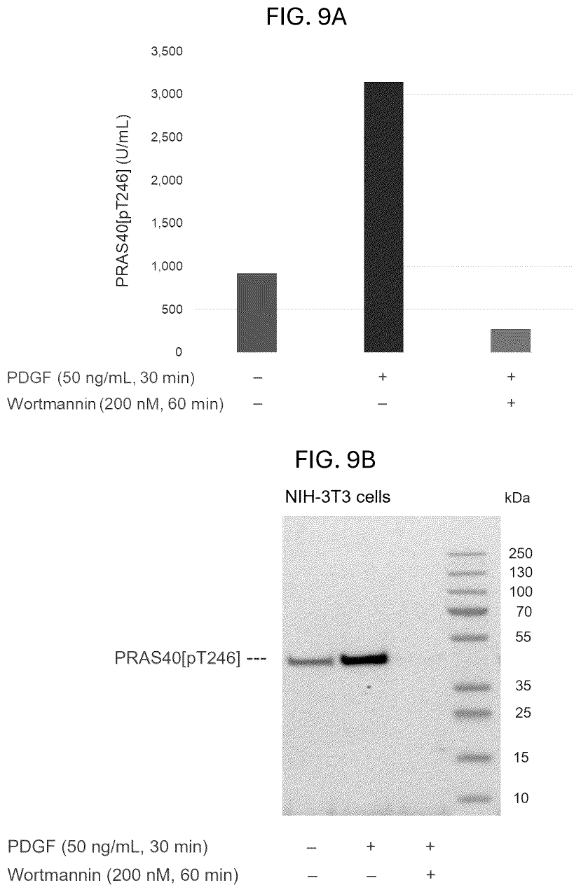

Comparison of protein analysis using Western blot and the dual reporter panel described in Example 3 was performed. Western blot and dual reporter panel assays to assess PRAS40[pT246] levels in lysed NIH-3T3 cells were performed. Equal amounts of protein (10 μg) were used for both assays. A high correlation between the two assay systems was observed (FIGS. 9A and 9B).

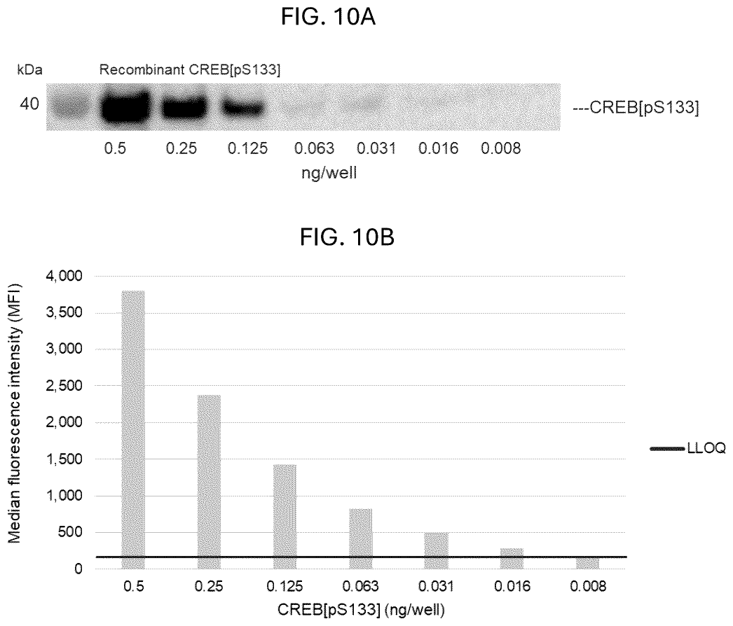

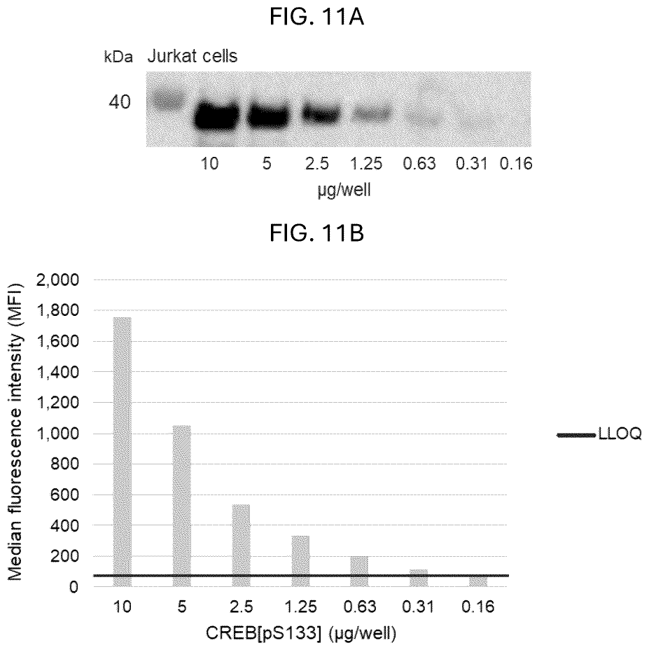

Sensitivity of a simplex assay and Western blotting was compared using a recombinant CREB[pS133]. The lowest concentration measured with Western blot was 0.031 ng/well, while the lowest concentration measured with the simplex assay was 0.016 ng/well (FIGS. 10A and 10B). This was also tested using Jurkat cell lysates. In this case, the lowest concentration measured with Western blot and the simplex assay was 0.31 ng/well, showing comparable performance (FIGS. 11A and 11B).

It will be apparent that the precise details of the methods or compositions described may be varied or modified without departing from the spirit of the described aspects of the disclosure. We claim all such modifications and variations that fall within the scope and spirit of the claims below.

We claim:

1. A method of detecting a plurality of analytes in a sample, comprising:
a) contacting the sample with a solid support comprising a capture reagent capable of specifically binding the plurality of analytes;
b) contacting the solid support with a plurality of detection antibodies or fragment thereof capable of specifically binding the plurality of analytes, wherein the plurality of detection antibodies or fragment thereof comprise at least:
i) a first detection antibody or fragment thereof capable of specifically binding a first analyte and comprising a first detectable label; and
ii) a second detection antibody or fragment thereof capable of specifically binding a second analyte and comprising a second detectable label;
c) contacting the solid support with a plurality of detection reagents, wherein the plurality of detection reagents comprise at least:
i) a first detection reagent capable of specifically binding to the first detectable label and comprising a first fluorescent label; and
ii) a second detection reagent capable of specifically binding to the second detectable label and comprising a second fluorescent label,
wherein at least one of the first and second detection reagents is an antibody or fragment thereof capable of specifically binding to the first or second detectable label; and
d) detecting the first fluorescent label and the second fluorescent label, thereby detecting the plurality of analytes.

2. The method of claim 1, wherein the capture reagent is an antibody or fragment thereof or a non-antibody capture reagent, optionally wherein the non-antibody capture reagent is an aptamer.

3. The method of claim 1, wherein:
the first analyte, the second analyte, or both are a protein;
the first analyte and the second analyte are different forms of the same analyte; and/or
the first analyte comprises a protein and the second analyte comprises a phosphorylated form of the same protein.

4. The method of claim 1, wherein:
(i) the first detectable label or the second detectable label comprises biotin, a dimethyl piperidine (DMP)-based chemical tag, digoxigenin, cucurbit[7]uril, O⁶-benzylguanine, a His tag comprising the amino acid sequence HHHHHH (SEQ ID NO:

1., a FLAG tag comprising the amino acid sequence DYKDDDDK (SEQ ID NO: 2), a Myc tag comprising the amino acid sequence EQKLISEEDL (SEQ ID NO: 3), a hemagglutinin tag comprising the amino acid sequence YPYDVPDYA (SEQ ID NO: 4, a fluorescent protein, HaloTag, or glutathione S-transferase; and/or
(ii) the first detection reagent or the second detection reagent comprises streptavidin, an antibody or fragment thereof that specific binds a dimethyl piperidine (DMP)-based chemical tag, an antibody or fragment thereof that specifically binds to digoxigenin, an antibody or fragment thereof that specifically binds to cucurbit[7]uril, SNAP tag (O⁶-alkylguanine-DNA alkyltransferase (AGT)), an antibody or fragment thereof that specifically binds to a His tag comprising the amino acid sequence HHHHHH (SEQ ID NO: 1), an antibody or fragment thereof that specifically binds to a FLAG tag comprising the amino acid sequence DYKDDDDK (SEQ ID NO: 2), an antibody or fragment thereof that specifically binds to a Myc tag comprising the amino acid sequence EQKLISEEDL (SEQ ID NO: 3), an antibody or fragment thereof that specifically binds to a hemagglutinin tag comprising the amino acid sequence YPYDVPDYA (SEQ ID NO: 4), an antibody or fragment thereof that specifically binds to a fluorescent protein, an antibody or fragment thereof that specifically binds to HaloTag, or an antibody or fragment thereof that specifically binds to glutathione S-transferase.

5. The method of claim 1, wherein:
the first detectable label and the second detectable label are different; and/or
the first detection reagent and the second detection reagent are different; and/or
the first fluorescent label and the second fluorescent label are different; and/or
the solid support comprises a bead.

6. The method of claim 1, further comprising contacting the sample with a plurality of solid supports, each comprising a different capture reagent, optionally wherein the plurality of solid supports comprises 2-500 solid supports, each comprising a different capture reagent.

7. The method of claim 1, wherein the first detectable label is biotin and the second detectable label is a dimethyl piperidine (DMP)-based chemical tag or is digoxigenin and wherein the first detection reagent is streptavidin and the second detection reagent is an antibody or fragment thereof capable of specifically binding to the DMP-based chemical tag or is an antibody or fragment thereof capable of specifically binding to digoxigenin; and/or
wherein the first fluorescent label is phycoerythrin and the second fluorescent label is a polymer dye, or wherein the first fluorescent label is a polymer dye and the second fluorescent label is phycoerythrin.

8. The method of claim 1, wherein the sample comprises cells, cell lysate, cell culture supernatant, extracellular vesicles, extracellular vesicle lysate, blood, serum, plasma, cerebrospinal fluid, urine, tissue, or tissue homogenate.

9. The method of claim 1, comprising:
a) contacting the sample with a solid support comprising a capture reagent capable of specifically binding to a protein involved in signal transduction;
b) contacting the solid support with a plurality of detection antibodies or fragment thereof capable of specifically binding the protein involved in signal transduction, wherein the plurality of detection antibodies or fragment thereof comprise at least:
i) a first detection antibody or fragment thereof capable of specifically binding the protein involved in signal transduction and a phosphorylated form of the protein involved in signal transduction and comprising biotin; and
ii) a second detection antibody or fragment thereof capable of specifically binding the phosphorylated protein involved in signal transduction and comprising a DMP-based chemical tag;
c) contacting the solid support with a plurality of detection reagents, wherein the plurality of detection reagents comprise at least:
i) a first detection reagent comprising streptavidin and a first fluorescent label; and
ii) a second detection reagent comprising an antibody or fragment thereof capable of specifically binding to the DMP-based chemical tag and comprising a second fluorescent label; and
d) detecting the first fluorescent label and the second fluorescent label, thereby detecting the plurality of analytes.

10. A kit comprising:
a solid support comprising a capture reagent capable of specifically binding an analyte;
a first detection antibody or fragment thereof capable of specifically binding the analyte and comprising a first detectable label;
a second detection antibody or fragment thereof capable of specifically binding the analyte and comprising a second detectable label;
a first detection reagent capable of specifically binding to the first detectable label and comprising a first fluorescent label; and
a second detection reagent capable of specifically binding to the second detectable label comprising a second fluorescent label,
wherein at least one of the first and second detection reagents is an antibody or fragment thereof capable of specifically binding to the first or second detectable label.

11. The kit of claim 10, wherein the capture reagent is an antibody or fragment thereof or a non-antibody capture reagent, optionally wherein the non-antibody capture reagent is an aptamer.

12. The kit of claim 10, wherein:
the first detectable label is biotin and the second detectable label is a dimethyl piperidine (DMP)-based chemical tag or digoxigenin and the first detection reagent is streptavidin and the second detection reagent is an antibody or fragment thereof capable of specifically binding to the DMP-based chemical tag or an antibody or fragment thereof capable of specifically binding to digoxigenin; and/or
the first fluorescent label is phycoerythrin and the second fluorescent label is a polymer dye, or wherein the first fluorescent label is a polymer dye and the second fluorescent label is phycoerythrin.

13. The kit of claim 10, wherein the solid support is a bead.

14. A method of detecting an analyte in two or more samples, comprising:
a) labeling a plurality of analytes in a first sample with a first detectable label to produce a labeled first sample and labeling a plurality of analytes in a second sample with a second detectable label to produce a labeled second sample;
a) contacting the labeled first sample and the labeled second sample with a solid support comprising a capture reagent capable of specifically binding at least one of the labeled analytes;
b) contacting the solid support with a plurality of detection reagents, wherein the plurality of detection reagents comprise at least:
i) a first detection reagent capable of specifically binding the first detectable label and comprising a first fluorescent label; and
ii) a second detection reagent capable of specifically binding the second detectable label and comprising a second fluorescent label, wherein at least one of the first and second detection reagents is an antibody or fragment thereof capable of specifically binding to the first or second tag; and
d) detecting the first fluorescent label and the second fluorescent label, thereby detecting the analyte from the first sample and the second sample.

15. The method of claim 14, wherein the capture reagent is an antibody or fragment thereof or a non-antibody capture reagent, optionally wherein the non-antibody capture reagent is an aptamer.

16. The method of claim 14, wherein the first sample is a reference sample and the second sample is a test sample.

17. The method of claim 14, wherein the analyte is a protein and/or the solid support is a bead.

18. The method of claim 14, wherein:
the first detectable label is biotin and the second detectable label is a dimethyl piperidine (DMP)-based chemical tag or digoxigenin and the first detection reagent is streptavidin and the second detection reagent is an antibody or fragment thereof capable of specifically binding to the DMP-based chemical tag or an antibody or fragment thereof capable of specifically binding to digoxigenin; and/or
the first fluorescent label is phycoerythrin and the second fluorescent label is a polymer dye, or wherein the first fluorescent label is a polymer dye and the second fluorescent label is phycoerythrin; and/or
the first fluorescent label and the second fluorescent label are different.

19. The method of claim 14, further comprising a plurality of solid supports, each comprising a different capture antibody, optionally wherein the plurality of solid supports comprises 2-500 solid supports, each comprising a different capture antibody.

20. The method of claim 14, further comprising comparing a level of the first fluorescent label and the second fluorescent label, thereby detecting a differential amount of the first analyte and the second analyte between the first and second samples.


SEQUENCE LISTING (4 sequences)

SEQ ID NO: 1 - protein, 6 aa, synthetic construct
HHHHHH

SEQ ID NO: 2 - protein, 8 aa, synthetic construct
DYKDDDDK

SEQ ID NO: 3 - protein, 10 aa, synthetic construct
EQKLISEEDL

SEQ ID NO: 4 - protein, 9 aa, synthetic construct
EPEDVPDYA